(19) United States
(12) Patent Application Publication
(10) Pub. No.: US 2011/0226272 A1
(43) Pub. Date: Sep. 22, 2011

(54) SHAVING KIT, ARTICLE OF COMMERCE AND A METHOD OF SHAVING COMPRISING A PERSONAL CARE COMPOSITION

(76) Inventors: Heather Lynn FOCHT, Hamilton, OH (US); Karl Shiqing Wei, Mason, OH (US); Edward Dewey Smith, III, Mason, OH (US); Christopher Dean Putman, West Chester, OH (US); Cheyne Pohlman Thomas, Independence, KY (US)

(21) Appl. No.: 12/904,842

(22) Filed: Oct. 14, 2010

Related U.S. Application Data
(63) Continuation of application No. 11/312,615, filed on Dec. 20, 2005, now abandoned.
Application No. 11/312,615 is a continuation of application No. 11/894,143, filed on Aug. 20, 2007.
Application No. 11/894,143 is a continuation of application No. 10/699,469, filed on Oct. 31, 2003, now Pat. No. 7,511,003.
(60) Provisional application No. 60/423,537, filed on Nov. 4, 2002.

(51) Int. Cl. A61Q 9/02; A61K 8/00; A45D 27/00; A45D 27/22

(52) U.S. Cl. 132/200; 424/73; 132/289

(57) ABSTRACT

A shaving kit comprising a personal care article is disclosed. The shaving kit comprises a package containing a personal care composition comprising a surfactant, water and at least about 15% of a hydrophobic moisturizing material and a plurality of razor blade cartridges or a shaving razor. An article of commerce is also disclosed that comprises a personal care article comprising a package containing a visually distinct, multiphase personal care composition and a set of in association with the personal care article. A method of shaving is also disclosed. The method comprises the steps of: wetting a skin surface; dispensing the multiphase personal care composition from the personal care article that comprises a package containing a visually distinct, multiphase personal care composition; contacting a skin surface with the personal care composition, and applying a shaving razor to the skin surface.

### CROSS REFERENCE TO RELATED APPLICATIONS

This application is a continuation of U.S. application Ser. No. 11/312,615 filed Dec. 20, 2005 and U.S. application Ser. No. 11/894,143 filed Aug. 20, 2007, which is a continuation of U.S. application Ser. No. 10/699,469 filed Oct. 31, 2003, now U.S. Pat. No. 7,511,003, which claims the benefit of U.S. Provisional Application Ser. No. 60/423,537 filed Nov. 4, 2002.

### FIELD OF THE INVENTION

The present invention relates to a shaving kit, an article of commerce and method of shaving comprising a personal care composition.

### BACKGROUND OF THE INVENTION

Consumers who shave typically use aerosol foaming compositions or gels. These foaming compositions provide a cushion and lubricate the skin during shaving. Unfortunately, some aerosol foaming compositions and gels have a tendency to dry the skin. Non-aerosol foaming compositions can be used to lather and lubricate the skin for wet shaving, yet may not provide a satisfactory degree of moisturization. Hydrophobic moisturizing materials, like petroleum, are well known skin lubricants and moisturizers. However, adding hydrophobic moisturizing materials to surfactant based non-aerosol compositions causes the compositions to lather poorly when hydrophobic moisturizing materials are introduced into the composition at levels sufficient to lubricate the skin for shaving. Moreover, compositions that largely comprise hydrophobic moisturizing material tend to clog the shaving razor causing poor performance. Therefore, even though aerosol composition and gels can dry their skin, consumers have continued to use these compositions out of habit and to avoid clogging their razor and blades.

Accordingly, the need still remains for a non-aerosol personal care composition that lathers well and lubricates the skin while providing improved skin conditioning benefits for use during shaving. Further, these non-aerosol personal care compositions should be packaged with razor blade cartridges and shaving razors to illustrate to the consumers the way the product can be used.

### SUMMARY OF THE INVENTION

The inventors believe that the present invention provides a kit that comprises a personal care composition that lathers well, and lubricates in combination with a razor blade cartridges, a shaving razor or instructions for shave, which illustrate to consumers that the compositions are suitable for use in shaving, as well as, suitable for use in cleansing, as a body wash.

The present invention relates to a shaving kit that comprises a personal care article and a plurality of razor blades. The personal care article comprises a package containing a personal care composition. The personal care composition comprises a surfactant, water and at least 15% of a hydrophobic moisturizing material.

The present invention further relates to a shaving kit that comprises a personal care article and a shaving razor. The personal care article comprises a package containing a personal care composition. The personal care composition comprises a surfactant, water and at least 15% of a hydrophobic moisturizing material.

The present invention also relates to an article of commerce that comprises a personal care article and set of instructions associated with the personal care article. The personal care article comprises a package containing a visually distinct, multiphase personal care composition. The visually distinct, multiphase personal care composition comprises a visually distinct cleansing phase and a visually distinct benefit phase. The visually distinct cleansing phase comprises a surfactant and water. The visually distinct cleansing phase and the visually distinct benefit phase are in physical contact within the package of the personal care article. The set of instructions comprises the steps of: wetting a skin surface; dispensing the visually distinct, multiphase personal care composition from the package; contacting the skin surface with the visually distinct, multiphase personal care composition; and applying a shaving razor to the skin surface.

The present invention further relates to a method of shaving. The method comprises the steps of: wetting a skin surface; dispensing the multiphase personal care composition from the personal care article comprising a package containing a visually distinct, multiphase personal care composition; contacting the skin surface with the personal care composition, and applying a shaving razor to the skin surface. The multiphase personal care composition comprises a visually distinct cleansing phase comprising a surfactant and water; and a visually distinct benefit phase. The cleansing phase and the benefit phase are in physical contact within the package of the personal care article.

### DETAILED DESCRIPTION OF THE INVENTION

The term “cleansing implement” as used herein is meant to device, or instrument use in performing the task of cleansing. Suitable cleansing implements include but are not limited to cleansing puff, sponge, brush, wash cloth, disposable cloth, and the like.

By the term “multiphase” or “multi-phase” as used herein, is meant that the phases of the present compositions occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phase” personal care compositions comprise at least two visually distinct phases which are present within the container as a visually distinct pattern. The pattern results from the combination of the “multi-phase” composition by a method of manufacture herein described. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled, and combinations thereof.

In a preferred embodiment, the striped pattern can be relatively uniform across the dimension of the package. Alternatively, the striped pattern can be uneven, i.e. wavy, or can be non-uniform in dimension. The striped pattern does not need to necessarily extend across the entire dimension of the package. The size of the stripes can be at least about 0.1 mm in width and 10 mm in length, preferably at least about 1 mm in width and at least 20 mm in length as measured from the package exterior. The phases can be various different colors, and/or include particles, glitter or pearlescent agents in at least one of the phases in order to offset its appearance from the other phase(s) present.

The term “package” includes any suitable container for a personal care compositions exhibiting a viscosity from about 1,500 centipoise (cP) to about 1,000,000 cP, of including but not limited to bottle, tottle, tube, jar, non-aerosol pump and mixtures thereof. The term package excludes the use of conventional aerosol dispensing containers or pressurized containers typically used for shave gels or shave foams.

The term “personal care composition” as used herein, refers to compositions intended for topical application to the skin.

The term “structured,” as used herein means having a rheology that confers stability on the multi-phase composition. The degree of structure is determined by the Yield Stress and Zero Shear Viscosity Method, described hereafter. When a phase is a structured phase, typically it has a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter. When a phase is a structured phase, it can also typically have a Zero Shear Viscosity of at least about 500 Pascal-seconds (Pa-s), preferably at least about 1,000 Pa-s.

As used herein “tottle” refers to a bottle which rests on neck or mouth which its contents are filled in and dispensed from, but it is also the end upon which the bottle is intended to rest or sit upon (e.g., the bottle's base) for storage by the consumer and/or for display on the store shelf (this bottle is referred to herein as a “tottle”). Typically, the closure on a tottle is flat or concave, such that the tottle, when stored, rests on the closure. Suitable tottles are described in the co-pending U.S. patent application Ser. No. 11/067443 filed on Feb. 25, 2005 to McCall, et al, entitled “Multi-phase Personal Care Compositions, Process for Making and Providing, and Article of Commerce.”

The term “visually distinct phase” as used herein, refers to a region of the multiphase personal care composition having one average composition, as distinct from another region having a different average composition, wherein the regions are visible to the unaided naked eye. This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase can also be constituted or re-constituted, collected, or separated into a bulk phase in order to observe its properties, e.g., by centrifugation, filtration or the like.

### Shaving Kit

The present invention relates to a shaving kit that comprises a personal care article and a plurality of razor blades. The personal care article comprises a package containing a personal care composition. The personal care composition comprises a surfactant, water and at least 15% of a hydrophobic moisturizing material.

The present invention relates to a shaving kit that comprises a personal care article and a shaving razor. The personal care article comprises a package containing a non-aerosol personal care composition. The personal care composition comprises a surfactant, water and at least 15% of a hydrophobic moisturizing material.

The shaving kit can further comprise a storage device for storing the shaving razor and plurality of razor blade cartridges. Suitable storage devices are those such as those disclosed in U.S. Pat. No. 6,415,517 issued to Charles Brigham Worrick, III on Jul. 9, 2002. The shaving kit can comprises a dispenser for dispensing a plurality of razor blade cartridges, such as those disclosed in U.S. Pat. No. 3,783,493 issued to Dawidowicz, et al on Jan. 8, 1974. The shaving razor, handle of the shaving razor, the razor blade cartridges, storage device, or dispenser may match in color the personal care composition or one of the phases of the personal care composition.

The shaving kit of can comprise a set of instructions comprising the steps of: wetting a skin surface; dispensing the personal care composition from the package; contacting the skin surface with the personal care composition; and applying a shaving razor to the skin surface. The set of instruction can further comprise the step of rubbing the personal care composition on the skin to form lather. The set of instructions further comprises the step of avoiding the use of a cleansing implement for contacting the skin surface with the personal case composition. The set of instructions can further comprise a cleansing step. The cleansing step comprises dispensing the personal care composition onto a cleansing implement and contacts a skin surface with the cleansing implement.

The shaving kit can comprise a one or more additional packages containing a personal care composition selected from the group consisting of a shampoo, conditioner, in-shower body moisturizer, body lotion, skin care composition, deodorant, antiperspirant, after shave lotion and mixtures thereof. The additional packages can be a size selected from the group consisting of full size, travel size, trial size and mixtures thereof. The shaving kit can further comprise a coupon, rebate, or advertisement. The coupon, rebate or advertisement is associated with a personal care products selected from the group consisting of a shampoo, conditioner, in-shower body moisturizer, body lotion, skin care composition, deodorant, antiperspirant, after shave lotion, shaving razor, razor blade cartridge, and mixtures thereof. The shaving kit can also comprise a grooming device selected from the group consisting of a cleansing cloth, disposable cleansing cloth, a pumice stone, a brush, a comb, a mirror, tweezers and mixtures thereof. The mirror can be a steam free, condensation proof, or non fogging mirror to facilitate shaving in the shower or bath. The components of the shaving kit may be shrink-wrapped together or packaged together in a plastic container.

### Article of Commerce

The present invention also relates to an article of commerce that comprises a personal care article and set of instructions associated with the personal care article. The personal care article comprises a package containing a visually distinct, multiphase personal care composition. The visually distinct, multiphase personal care composition that comprises a visually distinct cleansing phase and visually distinct benefit phase. The visually distinct cleansing phase comprises a surfactant and water. The visually distinct cleansing phase and the visually distinct benefit phase are in physical contact within the package of the personal care article. The set of instructions comprises the steps of: wetting a skin surface; dispensing the visually distinct, multiphase personal care composition from the package; contacting the skin surface with the visually distinct, multiphase personal care composition; and applying a shaving razor to the skin surface. The set of instruction can further comprise the step of rubbing the personal care composition on the skin to form lather. The set of instructions further comprises the step of avoiding the use of a cleansing implement for contacting the skin surface with the personal case composition. The set of instructions can further comprise a cleansing step. The cleansing step comprises dispensing the personal care composition onto a cleansing implement and contact a skin surface with the cleansing implement. The set of instructions may further state that the personal care composition is for use in the shower or bath. The visually distinct cleansing phase and the visually distinct benefit phase can form a pattern within the package. The package can be opaque, transparent, or translucent. The package can be a tottle.

### Method of Use

The present invention relates to a method of shaving, the method comprising the steps of: wetting a skin surface; dispensing the multiphase personal care composition from the personal care article comprising a package containing a visually distinct, multiphase personal care composition; contacting the skin surface with the personal care composition, and applying a shaving razor to the skin surface. The multiphase personal care composition comprises a visually distinct cleansing phase comprising a surfactant and water; and a visually distinct benefit phase. The cleansing phase and the benefit phase are in physical contact within the package of the personal care article. The method further comprises the step of rubbing the personal care composition on the skin to form lather. The method further comprises the step of avoiding the use of a cleansing implement for contacting the skin surface with the personal case composition. The method can further comprise a cleansing step. The cleansing step comprises dispensing the personal care composition onto a cleansing implement and contacting a skin surface with the cleansing implement. The method of shaving is preferably for use in the shower or bath.

### Personal Care Composition

The personal care composition can be comprises surfactant, water and at least about 15%, by weight of the personal care composition, of a hydrophobic moisturizing agent. The personal care composition of the present invention can comprises at least about 16.5%, by weight of personal care composition, of a hydrophobic moisturizing material, at least about 17%, by weight of personal care composition, of a hydrophobic moisturizing material, 17.5%, by weight of personal care composition, of a hydrophobic moisturizing material, at least about 20%, by weight of personal care composition, of a hydrophobic moisturizing material, at least about 24%, by weight of personal care composition, of a hydrophobic moisturizing material, at least about 28% hydrophobic moisturizing material; and at least about 30%, by weight of personal care composition, of a hydrophobic moisturizing material. It is believed that adding hydrophobic moisturizing material cushions, lubricates and moisturizes the skin surface for shaving. However, it also believed that personal care compositions with high levels of hydrophobic moisturizing material can cause shaving razors and blades to clog. Thus, the personal care composition of the present invention can comprises at less than about 70%, by weight of the personal care composition, of a hydrophobic moisturizing material, preferably less than about 50%, by weight of personal care composition, of a hydrophobic moisturizing material, and even more preferably less than about 40%, by weight of personal care composition, of a hydrophobic moisturizing material. Typically, the personal care composition will comprise from about 15% to about 50%, by weight of the personal care composition, of a hydrophobic moisturizing material, and preferably from about 17% to about 30%, by weight of the personal care composition, of a hydrophobic moisturizing material.

The hydrophobic moisturizing material can be selected from the group consisting of petrolatum, lanolin, hydrocarbon oils such as mineral oil, natural and synthetic waxes such as micro-crystalline waxes, paraffins, ozokerite, lanolin wax, lanolin alcohols, lanolin fatty acids, polyethylene, polybutene, polydecene and perhydrosqualene, volatile or non-volatile organosiloxanes and their derivatives such as dimethicones, cyclomethicones, alkyl siloxanes, polymethylsiloxanes and methylphenylpolysiloxanes, lanolin oil, esters such as isopropyl lanolate, acetylated lanolin, acetylated lanolin alcohols, lanolin alcohol linoleate, lanolin alcohol riconoleate natural and synthetic triglycerides such as castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and combinations thereof.

It is believed that the personal care compositions of the present invention would tend to clog typical aerosol containers. Thus, the personal care composition of the present invention will preferably be non-aerosol and will preferably be substantially free of propellants. The personal care composition will preferably comprise less than 1% propellant, more preferably 0.05% propellant, even more preferably 0.01% propellant and most preferably no propellant. These propellants can be any known to one skilled in the art. The personal care compositions of the present invention are substantially free of propellants selected from the group consisting of propane, isobutane and other petroleum distillates, nitrogen, carbon dioxide, dimethylether, methylethylether, methylene chloride, vinyl chloride and fluorochlorohydrocarbons. The latter include Freon 115 pentafluorochloroethane and Freon C-318, octafluorocyclobutane, gaseous chlorofluorinated C₁-C₂ hydrocarbon propellants, and mixtures thereof.

The personal care composition can be a multiphase composition that comprises a cleansing phase that comprises the surfactant and water and a benefit phase that comprises at least about 15% of a hydrophobic moisturizing material. The multiphase personal care composition of the present invention is typically extrudable or dispensible from a package. The multiphase personal care compositions typically exhibit a viscosity of from about 1,500 centipoise (cP) to about 1,000,000 cP, as measured by the Viscosity Method as described in copending application Ser. No. 10/841174 filed on May 7, 2004 titled “Multi-phase Personal Care Compositions.”

When evaluating a structured multiphase personal care composition, by the methods described herein, preferably each individual phase is evaluated prior to combining, unless otherwise indicated in the individual methodology. However, if the phases are combined, each phase can be separated by centrifugation, pipetting, filtering, washing, dilution, concentration, or combination thereof, and then the separate components or phases can be evaluated. Preferably, the separation means is chosen so that the resulting separated components being evaluated is not destroyed, but is representative of the component as it exists in the structured multiphase personal care composition, i.e., its composition and distribution of components therein is not substantially altered by the separation means. Generally, multiphase compositions comprise domains significantly larger than colloidal dimensions so that separation of the phases into the bulk is relatively easy to accomplish while retaining the colloidal or microscopic distribution of components therein. Preferably, the compositions of the present invention are rinse-off formulations, by which is meant the product is applied topically to the skin or hair and then subsequently the skin or hair is rinsed with water.

In a preferred embodiment of the present invention, the multiphase personal care composition comprises at least two visually distinct phases wherein a first phase is visually distinct from a second phase. Preferably, the visually distinct phases are packaged in physical contact with one another and are stable. Preferably, the visually distinct phases form a pattern.

### Phases:

The multiphase personal care compositions of the present invention comprise at least two visually distinct phases, wherein the composition can have a first structured phase, a second phase, a third phase, a fourth phase and so on. The ratio of a first phase to a second phase is preferably from about 90:10 to about 10:90, more preferably from about 80:20 to about 20:80, even more preferably from about 70:30 to about 30:70, still even more preferably from about 60:40 to about 40:60, even still even more preferably about 50:50. When a cleansing phase is present with a second phase the ratio of the cleansing phase to the second phase, by volume of the phases, is typically from about 99:1 to about 1:99, preferably from about, 90:10 to about 10:90, more preferably from about 80:20 to about 20:80, even more preferably from about 70:30 to about 30:70.

### Cleansing Phase:

The multiphase personal care composition of the present invention can comprise a cleansing phase. Preferably, the surfactant component comprises a mixture of surfactants. The multiphase personal care composition typically comprises from about 1% to about 99%, by weight of the composition, of the cleansing phase.

The cleansing phase preferably comprises a lathering surfactant or a mixture of lathering surfactants. The cleansing phase comprises surfactants suitable for application to the skin or hair. Suitable surfactants for use herein include any known or otherwise effective cleansing surfactant suitable for application to the skin, and which are otherwise compatible with the other essential ingredients in the multiphase personal care composition including water. These surfactants include anionic, nonionic, cationic, zwitterionic, amphoteric surfactants, soap, or combinations thereof. Preferably, anionic surfactant comprises at least 40% of the surfactant component.

The multiphase personal care composition preferably comprises a surfactants at concentrations ranging from about 2% to about 40%, more preferably from about 4% to about 25%, by weight of the first phase.

The cleansing phase is preferably comprised of a structured domain comprising surfactants. The structured domain enables the incorporation of high levels of benefit components in a separate phase that are not emulsified in the composition. In a preferred embodiment the structured domain is an opaque structured domain. The opaque structured domain is preferably a lamellar phase. The lamellar phase produces a lamellar gel network. The lamellar phase can provide resistance to shear, adequate yield to suspend particles and droplets and at the same time provides long term stability, since it is thermodynamically stable. The lamellar phase tends to have a higher viscosity thus minimizing the need for viscosity modifiers.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the surfactant component of the multiphase, personal care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in commonly owned U.S. Application Ser. No. 60/680,149 entitled “Structured Multi-phased Personal Cleansing Compositions Comprising Branched Anionic Surfactants” filed on May 12, 2004 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ pareth sulfate and sodium C₁₂₋₁₃ pareth-n sulfate.

Amphoteric surfactants suitable for use include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, and N-alkyltaurines. Amphoacetates and diamphoacetates, can also be used.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the multiphase, personal care composition include betaines, including cocoamidopropyl betaine.

Non-limiting examples of preferred nonionic surfactants for use herein are those selected form the group consisting of glucose amides, alkyl polyglucosides, sucrose cocoate, sucrose laurate, alkanolamides, ethoxylated alcohols and mixtures thereof. In a preferred embodiment the nonionic surfactant is selected from the group consisting of glyceryl monohydroxystearate, isosteareth-2, trideceth-3, hydroxystearic acid, propylene glycol stearate, PEG-2 stearate, sorbitan monostearate, glyceryl laurate, laureth-2, cocamide monoethanolamine, lauramide monoethanolamine, and mixtures thereof.

Mixtures of anionic surfactants can be used in some embodiments, including mixtures of linear and branched surfactants, and anionic surfactants with nonionic, amphoteric, and/or zwitterionic surfactants.

The electrolyte, if used, can be added per se to the multiphase personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium chloride, ammonium chloride, sodium or ammonium sulfate. The electrolyte is preferably added to the surfactant component of the composition in the amount of from about 0.1% to about 15% by weight, preferably from about 1% to about 6% by weight of the composition.

In one embodiment of the present invention, the multiphase, personal care composition comprises a surfactant component comprising a mixture of at least one nonionic surfactant, at least one anionic surfactant and at least one amphoteric surfactant, and an electrolyte. In another one embodiment, the surfactant can comprise a mixture of surfactants, water, at least one anionic surfactant, an electrolyte, and at least one alkanolamide.

### Benefit Phase:

The multiphase personal care compositions of the present invention can comprise a benefit phase. The benefit phase in the present invention is preferably anhydrous. The benefit phase typically comprises hydrophobic moisturizing materials. The benefit phase comprises from about 1% to about 100%, preferably at least about 15%, preferably at least about 17.5%, preferably at least about 20%, preferably at least about 24%, preferably at least about 30%, by weight of the benefit phase, of a hydrophobic moisturizing material. Hydrophobic moisturizing materials suitable for use in the present invention preferably have a Vaughan Solubility Parameter of from about 5 (cal/cm³)^{1.2} to about 15 (cal/cm³)^{1/2}, as defined by Vaughan in Cosmetics and Toiletries, Vol. 103. Non-limiting examples of hydrophobic moisturizing materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic compositions are preferably selected among those having defined rheological properties as described hereinafter, including selected Consistency value (K) and Shear Index (n). These preferred rheological properties are especially useful in providing the personal care compositions with lubrication of the skin surface for shaving and for improved deposition of hydrophobic moisturizing materials. The benefit phase has a Consistency Value (K) from about 20 to about 2,000 Pa-s, preferably from about 25 to about 500 Pa-s, more preferably from about 30 to about 450 Pa-s, still more preferably from about 30 to about 400 Pa-s and even still more preferably from about 30 to about 350 Pa-s. The benefit phase has a Shear Index from about 0.025 to about 0.99.

The benefit phase of the composition preferably can comprise one or more hydrophobic moisturizing materials, wherein at least 1% by weight of the hydrophobic moisturizing materials are selected from petrolatum, mineral oil, sunflower seed oil, alkyl siloxanes, polymethylsiloxanes and methylphenylpolysiloxanes, and combinations thereof. More preferably, at least about 20% by weight of the hydrophobic moisturizing materials are selected from the groups of petrolatum, mineral oil, paraffins, polyethylene, polydecene, dimethicones, alkyl siloxanes, lanolins. More preferably, at least about 50% by weight of the hydrophobic moisturizing materials are selected from the groups of petrolatum, mineral oil, paraffins, polydecene, dimethicones, alkyl siloxanes, lanolins.

Examples of suitable benefit phases and description of measuring the values of Consistency (K) and Shear Index (n) are described in U.S. patent application Ser. No. 10/665,670, Publication No. 2004/0057920 A1 entitled Striped liquid personal cleansing compositions containing a cleansing phase and a separate benefit phase” filed by Fact, et al. on Sep. 18, 2003, published on Apr. 4, 2004, U.S. patent application Ser. No. 10/699,469 Publication No. 2004/0092415 A1 entitled “Striped liquid personal cleansing compositions containing a cleansing phase and a separate benefit phase with improved stability” filed by Fact, et al. on Oct. 31, 2003, published on May 13, 2004 and U.S. patent application Ser. No. 10/837,214 Publication No. 2004/0219119 A1 entitled “Visually distinctive multiple liquid phase compositions” filed by Weir, et al. on Apr. 30, 2004, published on Nov. 18, 2004.

Density Modifiers To further improve stability under stress conditions such as high temperature and vibration, it is preferable to adjust the densities of the separate phases such that they are substantially equal. To achieve this, low density microspheres can be added to the cleansing phase of the personal care composition. The low density microspheres employed to reduce the overall density of the cleansing phase are particles having a density lower than 0.7 g/cm³, preferably less than 0.2 g/cm³. The low density microspheres generally have a diameter less than 200 μm, preferably less than 100 μm. Preferably, the density difference between the cleansing phase and the benefit phase is less than 0.15 g/cm³, more preferably, the density difference is less than 0.10 g/cm³, even more preferably, the density difference is less than 0.05 g/cm³.

The microspheres are produced from any appropriate inorganic or organic material, compatible with a use on the skin, that is, nonirritating and nontoxic.

Expanded microspheres are known, and may be obtained, for example, according to the processes described in Patents and Patent Applications EP-56219, EP-348372, EP-486080, EP-320473, EP-112807 and U.S. Pat. No. 3,615,972.

These microspheres may be produced from any nontoxic and non-irritant thermoplastic materials. These microspheres can be in the dry or hydrated state. Among hollow microspheres which can be used, special mention may be made of those marketed under the brand name EXPANCEL® (thermoplastic expandable microspheres) by the Akzo Nobel Company, especially those of DE (dry state) or WE (hydrated state) grade. Representative microspheres derived from an inorganic material, include, for instance, “Qcel® Hollow Microspheres” and “EXTENDOSPHERES™ Ceramic Hollow Spheres”, both available from the PQ Corporation.

The phases of the multiphase personal care composition, preferably the cleansing phase, can further comprise a polymeric phase structurant. The compositions of the present invention typically can comprise from about 0.05% to about 10%, preferably from about 0.1% to about 4%, of a polymeric phase structurant. Non-limiting examples of polymeric phase structurant include but is not limited to the following examples:, naturally derived polymers, synthetic polymers, crosslinked polymers, block copolymers, copolymers, hydrophilic polymers, nonionic polymers, anionic polymers, hydrophobic polymers, hydrophobically modified polymers, associative polymers, and oligomers.

The phase of the present compositions, preferably the cleansing phase, optionally can further comprise a liquid crystalline phase inducing structurant, which when present is at concentrations ranging from about 0.3% to about 15%, by weight of the phase. Suitable liquid crystalline phase inducing structurants include fatty acids (e.g. lauric acid, oleic acid, isostearic acid, linoleic acid) ester derivatives of fatty acids (e.g. propylene glycol isostearate, propylene glycol oleate, glyceryl isostearate) fatty alcohols, trihydroxystearin (available from Rheox, Inc. under the trade name THIXCIN® R). Preferably, the liquid crystalline phase inducing structurant is selected from lauric acid, trihydroxystearin, lauryl pyrrolidone, and tridecanol.

The multiphase personal care compositions of the present invention can additionally comprise an organic cationic deposition polymer in the one or more phases as a deposition aid for the benefit agents described herein. Suitable cationic deposition polymers for use in the compositions of the present invention contain cationic nitrogen-containing moieties such as quaternary ammonium moieties.

One or more of the phases of the multiphase personal care composition can comprise a variety of additional optional ingredients such as shiny particles, beads, exfoliating beads. Such optional ingredients are most typically those materials approved for use in cosmetics and that are described in reference books such as the CTFA Cosmetic Ingredient Handbook, Second Edition. The Cosmetic, Toiletries, and Fragrance Association, Inc. 1988, 1992.

Other non limiting examples of these optional ingredients include vitamins and derivatives thereof (e.g., ascorbic acid, vitamin E, tocopheryl acetate, and the like), sunscreens; thickening agents, preservatives for maintaining the anti microbial integrity of the cleansing compositions, anti-acne medicaments, antioxidants, skin soothing and healing agents such as aloe vera extract, allantoin and the like, chelators and sequestrants, skin lightening agents, and agents suitable for aesthetic purposes such as fragrances, essential oils, skin sensates, pigments, pearlescent agents and essential oils and fragrance.

### Test Methods

Yield Stress and Zero Shear Viscosity Method: The Yield Stress and Zero Shear Viscosity of a phase of the present composition, can be measured either prior to combining in the composition, or after combining in the composition by separating the phase by suitable physical separation means, such as centrifugation, pipetting, cutting away mechanically, rinsing, filtering, or other separation means.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress.

First a sample of the phase is obtained and placed in position on the rheometer base plate, the measurement geometry (upper plate) moving into position 1 mm above the base plate. Excess phase at the geometry edge is removed by scraping after locking the geometry. If the phase comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a phase has any particle larger than 5 mm in any dimension, the particles are removed prior to the measurement.

The determination is performed via the programmed application of a continuous shear stress ramp from 0.1 Pa to 1,000 Pa over a time interval of 5 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

### The Shear Index (n) and Consistency Value (K):

The Shear Index (n) and Consistency Value (K) are known and accepted means for reporting the viscosity profile of materials having a viscosity that varies with applied shear rate using a Power Law model. The term “Consistency value” or “K” as used herein is a measure of viscosity and is used in combination with Shear Index, to define viscosity for materials whose viscosity is a function of shear rate. The measurements of Consistency value and Shear Index are made at 25° C. The units for “Consistency value” or “K” are Pascal seconds. The units for “Shear Index” are dimensionless.

Viscosity of a phase can be measured by applying a shear stress and measuring the shear rate using a rheometer, such as a TA Instruments AR2000 (TA Instruments, New Castle, Del., USA 19720). Viscosity is determined at different shear rates in the following manner. First, the benefit phase is obtained. If there exists more than one distinct (immiscible, e.g.) benefit phase in the composition, such as for example a silicone oil phase and a hydrocarbon phase, they are preferably prepared separately and/or separated from each other, and evaluated separately from each other, although certain benefit phases which are mixtures such as emulsions can be evaluated as mixtures, in addition to evaluating the individual benefit phases individually.

For measurement, a 40 mm diameter parallel plate geometry with a gap of 1 mm is used unless there are particles greater than 0.25 mm, in which case a gap of 2 mm is used. The rheometer uses standard parallel plate conventions to report shear rate at the edge as shear rate of the test; and converts torque to stress using the factor 2/(πR³). Using a spatula, a sample comprising a small excess of the benefit phase is loaded onto the rheometer base plate which is at 25° C., the gap is obtained, and excess composition outside the top measurement geometry is removed, locking the top plate in position during the removal of excess sample. The sample is equilibrated to the base plate temperature for 2 minutes. A preshear step is performed comprising 15 seconds of shear at a shear rate of 50 inverse seconds (l/sec). As is known to one skilled in the art, the shear rate with a parallel plate geometry is expressed as the shear rate at the edge, which is also the maximum shear rate. After the preshear step, the measurement is performed, which comprises ramping the stress from 10 Pa to 1,000 Pa over a 2.0 minute interval at 25° C., while collecting 60 viscosity data points, in an evenly spaced linear progression. A shear rate of at least 500 l/seconds is obtained in the test, or the test is repeated with a fresh sample of the same component with a higher final stress value, maintaining the same rate of stress increase per time, until a shear rate of at least 500 l/sec is obtained during the measurement period. During the measurement, observe the sample to make certain the area under the top parallel plate is not evacuated of sample at any edge location during the measurement, or the measurement is repeated until a sample remains for the duration of the test. If after several trials a result cannot be obtained due to sample evacuation at the edge, the measurement is repeated leaving an excess reservoir of material at the edge (not scraping). If evacuation still cannot be avoided, a concentric cylinder geometry is used with a large excess of sample to avoid air pockets during loading. The results are fitted to the power law model by selecting only the data points between 25-500 l/sec shear rate, viscosity in Pa-s, shear rate in l/sec, and using a least squares regression of the logarithm of viscosity vs. the logarithm of shear rate to obtain values of K and n according to the Power Law equation:

μ=K(γ′)⁽ⁿ⁻¹⁾

The value obtained for the log-log slope is (n−1) where n is the Shear Index and the value obtained for K is the Consistency Value, expressed in units of in Pa-s.

### Method of Manufacture

The multiphase personal care compositions of the present invention can be prepared by any known or otherwise effective technique, suitable for making and formulating the desired multiphase product form. It is effective to combine toothpaste-tube filling technology with a spinning stage design. Additionally, the present invention can be prepared by the method and apparatus as disclosed in U.S. Pat. No. 6,213,166 issued to Thibiant, et al. on Apr. 10, 2001. The method and apparatus allows two or more compositions to be filled with a spiral configuration into a single container. The method requires that at least two nozzles be employed to fill the container. The container is placed on a static mixer and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. Specific non-limiting examples of such methods as they are applied to specific embodiments of the present invention are described in the following examples. Personal care composition can be manufactured according to the method disclosed in U.S. patent application Ser. No. 10/837,214 Publication No. 2004/0219119 A1 entitled “Visually distinctive multiple liquid phase compositions” filed by Weir, et al. on Apr. 30, 2004, published on Nov. 18, 2004.

If the multiphase personal care compositions contain patterns of varying colors it can be desirable to package these compositions in a transparent, or translucent package such that the consumer can view the pattern through the package.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

### Examples 1-3

The following examples described in Table 1 are non-limiting examples of personal care composition of the present invention.

TABLE 1
Personal Care Composition of the Present Invention

| | Example | Example | Example |
|---|---|---|---|
| | 1 | 2 | 3 |
| Ingredient | wt % | wt % | wt % |
| I. Cleansing Phase Composition | | | |
| Miracare SLB-365 (from Rhodia, Sodium | 47.4 | 47.4 | 47.4 |
| Trideceth Sulfate, Sodium Lauramphoacetate, | | | |
| Cocamide MEA) | | | |
| Polyquaterium 10 (UCare KG-30M) | 0.7 | — | — |
| Jaguar C-17 (from Rhodia) | | 0.7 | |
| Guar Hydroxypropyltrimonium Chloride | — | — | 0.7 |
| (N-Hance 3196 from Aqualon) | | | |
| PEG 90M (Polyox WSR 301 from Dow) | — | — | 0.2 |
| Glycerin | — | — | 0.6 |
| Sodium Chloride | 3.5 | 3.5 | 3.5 |
| Disodium EDTA | 0.15 | 0.1 | 0.1 |
| Glydant | 0.67 | 0.67 | 0.67 |
| Perfume | 1.37 | 2.0 | 1.44 |
| Expancel 091 DE 40 d30 (from | 0.4 | 0.4 | 0.4 |
| Expancel, Inc.) | | | |
| Water | Q.S. | Q.S. | Q.S. |
| (pH, QS with citric acid or NaOH) | (6.0) | (6.0) | (6.0) |
| II. Benefit phase Composition | | | |
| G2218 Petrolatum (WITCO) | — | — | 70 |
| Petrolatum (Superwhite Protopet from | 70 | 60 | — |
| WITCO) | | | |
| Mineral Oil (Hydrobrite 1000 PO White | 29.99 | 39.99 | 29.99 |
| MO from WITCO) | | | |
| Red 7 Cosmetic Pigment | 0.01 | 0.01 | 0.01 |
| Cleansing Phase:Benefit Phase Ratio | 80:20 | 50:50 | 70:30 |

Prepare the compositions described above by conventional formulation and mixing techniques. Prepare the cleansing phase composition by first adding citric acid into water at 1:3 ratio to form a citric acid premix. Prepare a Polyox/glycerin premix by adding Polyox into glycerin at 1:3 ratio. Add the following ingredients into the main mixing vessel in the following sequence with mixing: water, cationic polymer (Polyquaterium 10, Jaguar C-17, N-Hance 3196), and Expancel. Then, add Miracare SLB-365, sodium chloride, sodium benzoate, Disodium EDTA, and Glydant. Adjust the pH to 6.0 using citric acid premix. Add perfume and keep mixing until homogeneous.

Prepare the benefit phase by adding petrolatum into a mixing vessel. Heat the vessel to 190° F. Then, add mineral oil with agitation. Add cosmetic pigment and let the vessel cool down with slow agitation.

The cleansing and benefit phases are density matched to within 0.05 g/cm³. Package both phases at specified cleansing phase to benefit phase ratio using one of the methods of manufacturing described herein. The sample stage spins the bottle during filling process to create a marbled appearance.

### Examples 4-6

The following examples described in Table 2 are non-limiting examples of personal care compositions of the present invention.

TABLE 2
Personal Care Composition of the Present Invention

| Example | Example 4 | Example 5 | Example 6 |
|---|---|---|---|
| Ingredient | wt % | wt % | wt % |
| I. Cleansing Phase Composition | | | |
| Sodium Trideceth Sulfate (Cedepal | 8.1 | 8.1 | 8.1 |
| TD-407, Stepan) | | | |
| Sodium Lauryl Sulfate | 8.1 | 8.1 | 8.1 |
| Sodium Lauroamphoacetate (Miranol | 4.8 | 4.8 | 4.8 |
| L-32) | | | |
| Guar Hydroxypropyltrimonium | 0.6 | 0.6 | 0.6 |
| Chloride (N-Hance 3196 Aqualon) | | | |
| PEG 90M (Polyox WSR 301 from | 0.15 | 0.15 | 0.15 |
| Dow Chemical) | | | |
| Isosteareth-2 (Global Seven) | — | 1 | 2 |
| Trideceth-3 | 2 | 1 | — |
| Xanthan Gum (Keltrol 1000, | 0.22 | 0.22 | 0.22 |
| CP Kelco) | | | |
| Sodium Chloride | 4.75 | 4.75 | 4.75 |
| Disodium EDTA | 0.15 | 0.15 | 0.15 |
| Sodium Benzoate | 0.2 | 0.2 | 0.2 |
| Kathon CG | 0.03 | 0.03 | 0.03 |
| Perfume | 1.4 | 1.9 | 1.3 |
| Expancel 091 WE 40 d24 from | 0.33 | 0.33 | 0.33 |
| Expancel, Inc. | | | |
| Water | Q.S. | Q.S. | Q.S. |
| pH adjust to (use citric acid or NaOH) | 6.0 | 6.0 | 6.0 |
| II. Benefit phase Composition | | | |
| G2218 Petrolatum (WITCO) | — | — | 70 |
| Petrolatum (Superwhite Protopet, | — | 60 | — |
| WITCO) | | | |
| Petrolatum (Quidesa, Mexico) | 99.99 | — | — |
| Hydrobrite 1000 White Mineral Oil, | — | 39.99 | 29.99 |
| WITCO | | | |
| Red 7 Cosmetic Pigment | 0.01 | 0.01 | 0.01 |
| Cleansing Phase:Benefit Phase Ratio | 75:25 | 65:35 | 80:20 |

Prepare the compositions described above by conventional formulation and mixing techniques. First prepare a citric acid premix by adding citric acid into water at 1:3 ratio and a Polyox premix by adding Polyox WSR 301 and Keltrol 1000 to Trideceth-3 and Isosteareth-2. Then, add the following ingredients into the main mixing vessel with agitation: water, N-Hance 3196, Expancel, and Polyox premix. Then, add sodium trideceth sulfate, sodium lauroamphoacetate, sodium lauryl sulfate. Add sodium chloride, disodium EDTA, sodium benzoate, and Kathon CG. Adjust pH to 6.0 by adding citric acid premix. Then, add perfume and keep mixing until homogeneous.

Prepare the benefit phase by adding petrolatum into a mixing vessel. Heat the vessel to 190° F. Then, add mineral oil with agitation. Add cosmetic pigment and let the vessel cool down with slow agitation.

The cleansing and benefit phases are density matched to within 0.05 g/cm³. Package both phases at specified cleansing phase to benefit phase ratio using one of the methods of manufacturing described herein. The sample stage spins the bottle during filling process to create a marbled appearance.

### Examples 7-9

The following examples described in Table 3 are non-limiting examples of personal care compositions of the present invention.

TABLE 3
Personal Care Composition of the Present Invention

| Example | Example 7 | Example 8 | Example 9 |
|---|---|---|---|
| Ingredient | wt % | wt % | wt % |
| Sodium Trideceth Sulfate | 6.1 | 5.3 | 6.9 |
| (Cedepal TD-407) | | | |
| Sodium Lauryl Sulfate | 6.1 | 5.3 | 6.9 |
| Sodium Lauroamphoacetate | 3.6 | 3.1 | 4.1 |
| (Miranol L-32) | | | |
| Guar Hydroxypropyltrimonium | 0.45 | 0.39 | 0.51 |
| Chloride | | | |
| (N-Hance 3196 Aqualon) | | | |
| PEG 90M (Polyox WSR 301 | 0.11 | 0.1 | 0.13 |
| from Dow) | | | |
| Trideceth-3 | 1.5 | 1.3 | 1.7 |
| Xanthan Gum (Keltrol 1000, CP | 0.16 | 0.14 | 0.19 |
| Kelco) | | | |
| Sodium Chloride | 3.5 | 3.1 | 4.0 |
| Disodium EDTA | 0.11 | 0.1 | 0.13 |
| Sodium Benzoate | 0.15 | 0.13 | 0.17 |
| Kathon CG | 0.02 | 0.02 | 0.03 |
| Perfume | 1.1 | 1.1 | 1.1 |
| Expancel 091 WE 40 d24 from | 0.25 | 0.21 | 0.28 |
| Expancel, Inc. | | | |
| Petrolatum (Quidesa, Mexico) | 25 | 35 | 15 |
| Water | Q.S. | Q.S. | Q.S. |
| pH adjust to (use citric acid or | 6.0 | 6.0 | 6.0 |
| NaOH) | | | |

Prepare the compositions described above by conventional formulation and mixing techniques. First prepare a citric acid premix by adding citric acid into water at 1:3 ratio and a Polyox premix by adding Polyox WSR 301 and Keltrol 1000 to Trideceth-3 and Isosteareth-2. Then, add the following ingredients into the main mixing vessel with agitation: water, N-Hance 3196, Expancel, and Polyox premix. Then, add sodium trideceth sulfate, sodium lauroamphoacetate, sodium lauryl sulfate. Add sodium chloride, disodium EDTA, sodium benzoate, and Kathon CG. Adjust pH to 6.0 by adding citric acid premix. Then, add perfume and keep mixing until homogeneous. In a separate vessel, add Petrolatum and heat to 190 F. Add petrolatum into the main mixing vessel and keep mixing until homogeneous.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shaving kit comprising:
(a) personal care article comprising a package containing a personal care composition; said personal care composition comprising: a surfactant, water, and at least about 15%, by weight of personal care composition, of a hydrophobic moisturizing material; and
(b) a plurality of razor blade cartridges.

2. The shaving kit of claim 1, further comprising a shaving razor.

3. The shaving kit of claim 2, further comprising a storage device for storing said shaving razor and said plurality of said razor blades.

4. The shaving kit of claim 1, further comprising a dispenser for dispensing said plurality of razor blade cartridges.

5. The shaving kit of claim 1, further comprising a set of instructions comprising the steps of:
(a) wetting a skin surface;
(b) dispensing said personal care composition from said package;
(c) contacting said skin surface with said personal care composition; and
(d) applying a shaving razor to said skin surface.

6. The shaving kit of claim 5, wherein said set of instructions further comprises the step of avoiding the use of a cleansing implement for contacting said skin surface with said personal case composition.

7. The shaving kit of claim 1, further comprising one or more additional packages containing a personal care composition selected from the group consisting of a shampoo, conditioner, in-shower body moisturizer, body lotion, skin care composition, deodorant, antiperspirant, after shave lotion and mixtures thereof.

8. The shaving kit of claim 7, wherein said additional packages are a size selected from the group consisting of full size, travel size, trial size and mixtures thereof.

9. The shaving kit of claim 1, further comprising a coupon, rebate, or advertisement.

10. The shaving kit of claim 9, wherein said coupon, rebate or advertisement is associated with a personal care products selected from the group consisting of a shampoo, conditioner, in-shower body moisturizer, body lotion, skin care composition, deodorant, antiperspirant, after shave lotion, shaving razor, razor blade cartridges, and mixtures thereof.

11. The shaving kit of claim 1, further comprising a grooming device selected from the group consisting of a cleansing cloth, disposable cleansing cloth, a pumice stone, a brush, a comb, a mirror, tweezers and mixtures thereof.

12. The shaving kit of claim 1, wherein said hydrophobic moisturizing material is selected from the group consisting of petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and mixtures thereof.

13. The shaving kit of claim 1, wherein said personal care composition comprises at least about 17.5%, by weight of personal care composition, of a hydrophobic moisturizing material.

14. The shaving kit of claim 1, wherein said personal care composition comprises at least about 30%, by weight of personal care composition, of a hydrophobic moisturizing material.

15. The shaving kit of claim 1, wherein said personal care composition is a multiphase composition comprising a cleansing phase comprising said surfactant and said water and a benefit phase comprising at least about 15% of a hydrophobic moisturizing material.

16. A shaving kit comprising:
(a) personal care article comprising a package containing a personal care composition;
said personal care composition comprising: a surfactant, water, and at least about 15%, by weight of personal care composition, of a hydrophobic benefit material.
(b) a shaving razor.

17. The shaving kit of claim 16, further comprising a plurality of razor blade cartridges.

18. An article of commerce comprising:
(a) personal care article comprising a package containing a visually distinct, multiphase personal care composition; said visually distinct, multiphase personal care composition comprising:
i. a visually distinct cleansing phase comprising a surfactant and water;
ii. a visually distinct benefit phase;
wherein said cleansing phase and said benefit phase are in physical contact within said package; and
(b) a set of instructions in association with said personal care article; said set of instructions comprising the steps of:
1. wetting a skin surface;
2. dispensing said visually distinct, multiphase personal care composition from said package;
3. contacting a skin surface with said visually distinct, multiphase personal care composition; and
4. applying a shaving razor to said skin surface.

19. The article of commerce of claim 18, wherein said set of instructions further comprises the step of rubbing the personal care composition on the skin to form lather.

20. The article of commerce of claim 18, wherein said set of instructions further comprises the step of avoiding the use of a cleansing implement for contacting said skin surface with said visually distinct, multiphase personal case composition.

21. The article of commerce of claim 18, wherein said set of instructions further comprises a cleansing step; wherein said cleansing step comprises applying said visually distinct, multiphase personal care composition on a cleansing implement and contacting a skin surface with said cleansing implement.

22. A method of shaving comprising the steps of :
1. wetting a skin surface:
2. dispensing a multiphase personal care composition from a personal care article comprising a package containing said multiphase personal care composition;
wherein said multiphase personal care composition comprises:
(a) a visually distinct cleansing phase comprising a surfactant and water; and
(b) a visually distinct benefit phase;
wherein said cleansing phase and said benefit wherein said benefit phase are in physical contact within said package; and
3. contacting the skin surface with said multiphase personal care composition, and
4. applying a shaving razor to said skin surface.

23. The method of shaving of claim 22, wherein said method further comprises rubbing the personal care composition on the skin to form lather.

24. The method of shaving of claim 22, wherein said method further comprises the step of avoiding the use of a cleansing implement for contacting said skin surface with said multiphase personal case composition.

25. The method of shaving of claim 22, further comprising a cleansing step; wherein said cleansing step comprises applying said multiphase personal care composition on a cleansing implement and contacting a skin surface with said cleansing implement.
